(19) United States
(12) Patent Application Publication
(10) Pub. No.: US 2025/0091987 A1
(43) Pub. Date: Mar. 20, 2025

(54) Wash-Active Compounds

(71) Applicant: Henkel AG & Co. KGaA, Duesseldorf (DE)

(72) Inventors: Caroline Hartwig, Duesseldorf (DE); Peter Schmiedel, Duesseldorf (DE); Mareile Job, Leverkusen (DE)

(21) Appl. No.: 18/959,090

(22) Filed: Nov. 25, 2024

Related U.S. Application Data
(63) Continuation of application No. PCT/EP2023/058646, filed on Apr. 3, 2023.

(30) Foreign Application Priority Data
May 30, 2022 (DE) 102022205390.7

(51) Int. Cl. C07C 309/49; C07C 303/22; C11D 3/34

(52) U.S. Cl. CPC C07C 309/49; C07C 303/22; C11D 3/349; C11D 2111/12; C11D 2111/14

(57) ABSTRACT

Disclosed is a compound of the general formula (1) R—X—SO₃—HN+(RₐR_{b}R_{c}) (1), wherein i) R is selected from the group of C₆ to C₂₀ alkyl groups; ii) X is selected from the group a) C₆H₄, b) (OC2H4)nO, in which n=2 to 8, c) C(O)—N(CH₃)CH₂CH₂, and d) R1O(O)CCH2(R1O(O)C)CH, in which R¹═C₆ to C₂₀ alkyl; iii) at least two of the groups Rₐ, R_{b}, and R_{c} are selected independently of each other from the group consisting of C₃ to C₁₂ alkyl and C₃ to C₁₂ hydroxyalkyl groups. Also disclosed are detergents containing the compound, and washing and cleaning methods using the compound, and methods for producing same.

### FIELD OF THE INVENTION

The present invention relates to a wash-active compound. The application furthermore relates to detergents and cleaning agents that contain this wash-active compound, to processes for preparing the wash-active compound, and to detergents and cleaning processes using the wash-active compound.

### BACKGROUND OF THE INVENTION

Continuously changing requirements are placed on the forms of manufacture and supply of washing and cleaning agents. A trend relevant to the preparation of detergents and cleaning agents is their concentration. In addition to higher consumer acceptance due to simplified handling, the background of this development are, in particular, sustainability aspects—for example, in relation to transport volumes and costs and the quantity of packaging materials used.

The concentration of modern detergents and cleaning agents, in particular modern liquid detergents, generally influences their optical and rheological properties, has effects on the storage stability of these agents, and can influence their cleaning performance, in particular when the high concentration of the active substances leads to incompatibilities.

In addition, concentrating detergents and cleaning agents usually involves the use of organic solvents. These solvents improve the physical stability of the detergents and cleaning agents, but are often characterized by only low washing or cleaning activity, as a result of which they should be added in the smallest possible quantities, not least for sustainability reasons. However, the use of aqueous or aqueous-organic solvent systems in the production of concentrated liquid detergents cannot yet be avoided with conventional wash- or cleaning-active ingredients.

European patent application EP 3 670 638 A1 describes the use of ionic liquids in combination with conventional aqueous or aqueous-organic solvent systems as a component of detergents or cleaning agents.

U.S. Pat. No. 7,737,102 B2 describes cleaning-active ionic liquids based upon a combination of anionic surfactants and specific amine oxides.

Against this background, the application was based upon the object of providing wash- and cleaning-active substances that enable the preparation of visually appealing, concentrated, flowable detergent preparations. It should be possible to produce the active ingredients in a simple and efficient manner, and they should have good storage capacity, and be characterized in particular by good cleaning results.

This object was achieved by a new class of surfactant active ingredients.

### BRIEF SUMMARY OF THE INVENTION

The application relates to a compound of general formula (1) and a method of use and producing the compound.

R—X—SO₃⁻HN⁺(RₐR_{b}R_{c}) (1),
wherein
i) R is selected from the group of C₆ to C₂₀ alkyl functional groups;
ii) X is selected from the group
   a) C₆H₄
   b) (OC₂H₄)ₙO where n=2 to 8
   c) C(O)—N(CH₃)CH₂CH₂
   d) R¹O(O)CCH₂(R¹O(O)C)CH, in which R¹═C₆ to C₂₀ alkyl
iii) at least two of the functional groups Rₐ, R_{b}, and R_{c} are selected independently of each other from the group consisting of C₃ to C₁₂ alkyl and C₃ to C₁₂ hydroxyalkyl functional groups.

### DETAILED DESCRIPTION OF THE INVENTION

A first subject matter of the application is a compound of general formula (1) R—X—SO₃⁻HN⁺(RₐR_{b}R_{c}) (1), wherein
i) R is selected from the group of C₆ to C₂₀ alkyl functional groups;
ii) X is selected from the group
   a) C₆H₄
   b) (OC₂H₄)ₙO where n=2 to 8
   c) C(O)—N(CH₃)CH₂CH₂
d) R¹O(O)CCH₂(R¹O(O)C)CH, in which R¹═C₆ to C₂₀ alkyl iii) at least two of the functional groups Rₐ, R_{b}, and R_{c} are selected independently of each other from the group consisting of C₃ to C₁₂ alkyl and C₃ to C₁₂ hydroxyalkyl functional groups.

Surprisingly, it was found that surface-active ionic liquids can be obtained by neutralizing specific anionic surfactant acids containing sulfonic acid groups using dialkyl- or trialkyl- or -hydroxyalkylamines. The amorphous, transparent liquids are suitable, for example, for the production of solvent-free liquid detergents with active ingredient contents of up to 100 wt. %.

A particularly preferred compound of general formula (1) is based upon anionic detergents from the group of alkylbenzene sulfonic acids. Particularly preferred compounds of general formula (1) are therefore characterized in that
i) R is selected from the group of C₁₀ to C₁₃ alkyl functional groups;
ii) X is selected from group C₆H₄
iii) at least two of the functional groups Rₐ, R_{b}, and R_{c} are selected independently of each other from the group consisting of C₃ to C₁₂ alkyl and C₃ to C₁₂ hydroxyalkyl functional groups.

Functional groups R selected from the group of linear C₁₀ to C₁₃ alkyl groups, secondary C₁₀ to C₁₃ alkyl functional groups and in particular linear secondary C₁₀ to C₁₃ alkyl functional groups have proven to be particularly advantageous. Very particular preference is given to functional groups R from the group of linear C₁₂H₂₅ alkyl functional groups, in particular the linear secondary C₁₂H₂₅ alkyl functional groups.

In alternative embodiments, the sulfonic acid on which the compound of general formula (1) is based can be varied.

A first alternative forms compounds of general formula (1), wherein
i) R is selected from the group of C₁₂ to C₂₀ alkyl functional groups;
ii) X is selected from the group selected from the group (OC₂H₄)ₙO where n=1 to 8
iii) at least two of the functional groups Rₐ, R_{b}, and R_{c} are selected independently of each other from the group consisting of C₃ to C₁₂ alkyl and C₃ to C₁₂ hydroxyalkyl functional groups.

A further alternative comprises compounds of general formula (1), wherein
i) R is selected from the group of C₇ to C₁₇ alkyl functional groups;
ii) X is selected from the group C(O)—N(CH₃)CH₂CH₂;
iii) at least two of the functional groups Rₐ, R_{b}, and R_{c} are selected independently of each other from the group consisting of C₃ to C₁₂ alkyl and C₃ to C₁₂ hydroxyalkyl functional groups.

The third alternative represents compounds of general formula (1), wherein
i) R is selected from the group of C₇ to C₁₇ alkyl functional groups;
ii) X is selected from the group O(O)CCH₂(RO(O)C)CH where R═C₆ to C₁₇ alkyl;
iii) at least two of the functional groups Rₐ, R_{b}, and R_{c} are selected independently of each other from the group consisting of C₃ to C₁₂ alkyl and C₃ to C₁₂ hydroxyalkyl functional groups.

In addition to the sulfonic acid group-containing anionic surfactant acids, the compounds of general formula (1) furthermore comprise specific dialkyl- or trialkyl- or -hydroxyalkylamines.

In a first embodiment, at least two of the functional groups Rₐ, R_{b}, and R_{c} are selected independently of each other from the group consisting of C₃ to C₁₂ alkyl functional groups, preferably from the group consisting of C₃ to C₆ alkyl functional groups. In particular, compounds of general formula (1) in which the three functional groups Rₐ, R_{b}, and R_{c} are selected independently of each other from the group consisting of C₃ to C₁₂ alkyl functional groups, preferably from the group consisting of C₃ to C₆ alkyl functional groups, are particularly preferred.

Some preferred compounds of general formula R—X—SO₃⁻HN⁺(RₐR_{b}R_{c}), in which
X is selected from the group
a) C₆H₄
b) (OC₂H₄)ₙO where n=1 to 8
c) C(O)—N(CH₃)CH₂CH₂
d) O(O)CCH₂(R¹O(O)CH, in which R¹═C₆ to C₂₀ alkyl,
can be found in the following table:

| | R | Rₐ/R_{b} | R_{c} |
|---|---|---|---|
| 1a | C₆ to C₂₀ alkyl | C₃ to C₁₂ alkyl | H |
| 2a | C₆ to C₂₀ alkyl | C₃ to C₆ alkyl | H |
| 3a | C₆ to C₂₀ alkyl | C₃ alkyl | H |
| 4a | C₆ to C₂₀ alkyl | n-C₃ alkyl | H |
| 5a | C₆ to C₂₀ alkyl | iso-C₃ alkyl | H |
| 6a | C₆ to C₂₀ alkyl | C₄ alkyl | H |
| 7a | C₆ to C₂₀ alkyl | n-C₄ alkyl | H |
| 8a | C₆ to C₂₀ alkyl | iso-C₄ alkyl | H |
| 9a | C₆ to C₂₀ alkyl | sec-C₄ alkyl | H |
| 10a | C₁₂ alkyl | C₃ to C₁₂ alkyl | H |
| 11a | C₁₂ alkyl | C₃ to C₆ alkyl | H |
| 12a | C₁₂ alkyl | C₃ alkyl | H |
| 13a | C₁₂ alkyl | n-C₃ alkyl | H |
| 14a | C₁₂ alkyl | iso-C₃ alkyl | H |
| 15a | C₁₂ alkyl | C₄ alkyl | H |
| 16a | C₁₂ alkyl | n-C₄ alkyl | H |
| 17a | C₁₂ alkyl | iso-C₄ alkyl | H |
| 18a | C₁₂ alkyl | sec-C4 alkyl | H |
| 19a | C₆ to C₂₀ alkyl | C₃ to C₁₂ alkyl | C₃ to C₁₂ alkyl |
| 20a | C₆ to C₂₀ alkyl | C₃ to C₆ alkyl | C₃ to C₆ alkyl |
| 21a | C₆ to C₂₀ alkyl | C₃ alkyl | C₃ alkyl |
| 22a | C₆ to C₂₀ alkyl | n-C₃ alkyl | n-C₃ alkyl |
| 23a | C₆ to C₂₀ alkyl | iso-C₃ alkyl | iso-C₃ alkyl |
| 24a | C₆ to C₂₀ alkyl | C₄ alkyl | C₄ alkyl |
| 25a | C₆ to C₂₀ alkyl | n-C₄ alkyl | n-C₄ alkyl |
| 26a | C₆ to C₂₀ alkyl | iso-C₄ alkyl | iso-C₄ alkyl |
| 27a | C₆ to C₂₀ alkyl | sec-C₄ alkyl | sec-C₄ alkyl |
| 28a | C₁₂ alkyl | C₃ to C₁₂ alkyl | C₃ to C₁₂ alkyl |
| 29a | C₁₂ alkyl | C₃ to C₆ alkyl | C₃ to C₆ alkyl |
| 30a | C₁₂ alkyl | C₃ alkyl | C₃ alkyl |
| 31a | C₁₂ alkyl | n-C₃ alkyl | n-C₃ alkyl |
| 32a | C₁₂ alkyl | iso-C₃ alkyl | iso-C₃ alkyl |
| 33a | C₁₂ alkyl | C₄ alkyl | C₄ alkyl |
| 34a | C₁₂ alkyl | n-C₄ alkyl | n-C₄ alkyl |
| 35a | C₁₂ alkyl | iso-C₄ alkyl | iso-C₄ alkyl |
| 36a | C₁₂ alkyl | sec-C₄ alkyl | sec-C₄ alkyl |

In a second embodiment, at least two of the functional groups Rₐ, R_{b}, and R_{c} are selected independently of each other from the group consisting of C₃ to C₁₂ hydroxyalkyl functional groups, preferably from the group consisting of C₃ to C₆ hydroxyalkyl functional groups. In particular, compounds of general formula (1) in which the three functional groups Rₐ, R_{b}, and R_{c} are selected independently of each other from the group consisting of C₃ to C₁₂ hydroxyalkyl functional groups, preferably from the group consisting of C₃ to C₆ hydroxyalkyl functional groups, are particularly preferred.

Particularly advantageous properties are exhibited by compounds of the general formula (1) in which the three functional groups Rₐ, R_{b}, and R_{c} each represent a (CH₂CH(OH)CH₃) hydroxyalkyl functional group.

Very particularly preference is given to a compound of general formula (1) in which
i) R represents a linear C₁₂H₂₅ alkyl functional group, preferably a linear secondary C₁₂H₂₅ alkyl functional group;
ii) X is selected from group C₆H₄
iii) Rₐ, R_{b}, and R_{c} each represent a (CH₂CH(OH)CH₃) hydroxyalkyl functional group.

Some particularly preferred compounds of general formula R—X—SO₃⁻HN⁺(RₐR_{b}R_{c}), in which
X is selected from the group
a) C₆H₄
b) (OC₂H₄)ₙO where n=1 to 8
c) C(O)—N(CH₃)CH₂CH₂
d) O(O)CCH₂(R¹O(O)C)CH, in which R¹═C₆ to C₂₀ alkyl,
can be found in the following table:

| | R | Rₐ/R_{b} | R_{c} |
|---|---|---|---|
| 1b | C₆ to C₂₀ alkyl | C₃ to C₁₂ hydroxyalkyl | H |
| 2b | C₆ to C₂₀ alkyl | C₃ to C₆ hydroxyalkyl | H |
| 3b | C₆ to C₂₀ alkyl | C₃ hydroxyalkyl | H |
| 4b | C₆ to C₂₀ alkyl | n-C₃ hydroxyalkyl | H |
| 5b | C₆ to C₂₀ alkyl | iso-C₃ hydroxyalkyl | H |
| 6b | C₆ to C₂₀ alkyl | C₄ hydroxyalkyl | H |
| 7b | C₆ to C₂₀ alkyl | n-C₄ hydroxyalkyl | H |
| 8b | C₆ to C₂₀ alkyl | iso-C₄ hydroxyalkyl | H |
| 9b | C₆ to C₂₀ alkyl | sec-C₄ hydroxyalkyl | H |
| 10b | C₁₂ alkyl | C₃ to C₁₂ hydroxyalkyl | H |
| 11b | C₁₂ alkyl | C₃ to C₆ hydroxyalkyl | H |
| 12b | C₁₂ alkyl | C₃ hydroxyalkyl | H |
| 13b | C₁₂ alkyl | n-C₃ hydroxyalkyl | H |
| 14b | C₁₂ alkyl | iso-C₃ hydroxyalkyl | H |
| 15b | C₁₂ alkyl | C₄ hydroxyalkyl | H |
| 16b | C₁₂ alkyl | n-C₄ hydroxyalkyl | H |
| 17b | C₁₂ alkyl | iso-C₄ hydroxyalkyl | H |
| 18b | C₁₂ alkyl | sec-C₄ hydroxyalkyl | H |
| 19b | C₆ to C₂₀ alkyl | C₃ to C₁₂ hydroxyalkyl | C₃ to C₁₂ hydroxyalkyl |
| 20b | C₆ to C₂₀ alkyl | C₃ to C₆ hydroxyalkyl | C₃ to C₆ hydroxyalkyl |
| 21b | C₆ to C₂₀ alkyl | C₃ hydroxyalkyl | C₃ hydroxyalkyl |
| 22b | C₆ to C₂₀ alkyl | n-C₃ hydroxyalkyl | n-C₃ hydroxyalkyl |
| 23b | C₆ to C₂₀ alkyl | iso-C₃ hydroxyalkyl | iso-C₃ hydroxyalkyl |
| 24b | C₆ to C₂₀ alkyl | C₄ hydroxyalkyl | C₄ hydroxyalkyl |
| 25b | C₆ to C₂₀ alkyl | n-C₄ hydroxyalkyl | n-C₄ hydroxyalkyl |
| 26b | C₆ to C₂₀ alkyl | iso-C₄ hydroxyalkyl | iso-C₄ hydroxyalkyl |
| 27b | C₆ to C₂₀ alkyl | sec-C₄ hydroxyalkyl | sec-C₄ hydroxyalkyl |
| 28b | C₁₂ alkyl | C₃ to C₁₂ hydroxyalkyl | C₃ to C₁₂ hydroxyalkyl |
| 29b | C₁₂ alkyl | C₃ to C₆ hydroxyalkyl | C₃ to C₆ hydroxyalkyl |
| 30b | C₁₂ alkyl | C₃ hydroxyalkyl | C₃ hydroxyalkyl |
| 31b | C₁₂ alkyl | n-C₃ hydroxyalkyl | n-C₃ hydroxyalkyl |
| 32b | C₁₂ alkyl | iso-C₃ hydroxyalkyl | iso-C₃ hydroxyalkyl |
| 33b | C₁₂ alkyl | C₄ hydroxyalkyl | C₄ hydroxyalkyl |
| 34b | C₁₂ alkyl | n-C₄ hydroxyalkyl | n-C₄ hydroxyalkyl |
| 35b | C₁₂ alkyl | iso-C₄ hydroxyalkyl | iso-C₄ hydroxyalkyl |
| 36b | C₁₂ alkyl | sec-C₄ hydroxyalkyl | sec-C₄ hydroxyalkyl |

Very particular preference is given to a compound of formula C₁₂H₂₅—C₆H₄—SO₃⁻HN⁺(CH₂CH(OH)CH₃)₃.

As stated at the outset, the compounds of general formula (1) are particularly suitable as components of detergent or cleaning agents, in particular liquid detergent or cleaning agents, due to their physical properties. Corresponding detergent or cleaning agents containing a compound of general formula (1) are therefore further subject matter of this application.

A washing agent is a composition which is suitable for cleaning or caring for textiles and contains at least one textile-cleaning or textile-care active substance.

Examples of textile-cleaning active substances are
surfactants, in particular nonionic or anionic surfactants,
builders, such as phosphonates or water-softening polymers,

Examples of textile-care active substances are
fragrances, in particular encapsulated fragrances,
fabric softeners, in particular cationic surfactants and cationic polymers,
dyes, in particular textile coloring agents.

The detergents are preferably free of fluorine-containing hydrocarbon solvents.

A further subject matter of the application is a process for washing textiles, in the course of which a compound of the general formula (1) or a detergent containing a compound of the general formula (1) is introduced into a washing liquor. The washing liquor is preferably aqueous and free of fluorine-containing hydrocarbon solvents.

A cleaning agent is a composition which is suitable for cleaning and caring for hard surfaces and contains at least one surface-cleaning or surface-care active substance.

Examples of surface-cleaning active substances are
surfactants, in particular nonionic or anionic surfactants,
builders, such as phosphonates or water-softening polymers,

Examples of surface-care active substances are
corrosion inhibitors, in particular zinc salts,
hydrophobizing agents, in particular polymeric hydrophobizing agents.

The cleaning agents are preferably free of fluorine-containing hydrocarbon solvents.

A process for cleaning surfaces, in the course of which a compound of general formula (1) or a cleaning agent containing a compound of general formula (1) is introduced into a cleaning liquor, is an additional subject matter of this application. The cleaning liquor is preferably aqueous and free of fluorine-containing hydrocarbon solvents.

The composition of some preferred liquid detergent or cleaning agents can be found in the following tables (amounts given in wt. % based upon the total weight of the preparation, unless otherwise indicated). The listed detergent or cleaning agents are free of fluorine-containing hydrocarbon solvents.

| | Formula 1 | Formula 2 | Formula 3 | Formula 4 |
|---|---|---|---|---|
| A compound of general | 5 to 80 | 10 to 70 | 15 to 65 | 20 to 60 |
| formula (1) | | | | |
| Non-ionic surfactant | 5 to 50 | 10 to 45 | 15 to 40 | 20 to 35 |
| Misc. | up to 100 | up to 100 | up to 100 | up to 100 |
| | Formula 6 | Formula 2 | Formula 8 | Formula 9 |
| A compound of general | 5 to 80 | 10 to 70 | 15 to 65 | 20 to 60 |
| formula (1) | | | | |
| Non-ionic surfactant | 5 to 50 | 10 to 45 | 15 to 40 | 20 to 35 |
| Polyalkoxylated amine ¹⁾ | 0.5 to 10 | 1 to 8 | 1 to 8 | 3 to 6 |
| Misc. | up to 100 | up to 100 | up to 100 | up to 100 |

| | Formula 11 | Formula 12 | Formula 13 | Formula 14 |
|---|---|---|---|---|
| A compound of general | 5 to 80 | 10 to 70 | 15 to 65 | 20 to 60 |
| formula (1) | | | | |
| Non-ionic surfactant | 5 to 50 | 10 to 45 | 15 to 40 | 20 to 35 |
| Polyalkoxylated | 0.5 to 10 | 1 to 8 | 1 to 8 | 3 to 6 |
| polyalkyleneimine ²⁾ | | | | |
| Misc. | up to 100 | up to 100 | up to 100 | up to 100 |
| | Formula 16 | Formula 17 | Formula 18 | Formula 19 |
| A compound of general | 5 to 80 | 10 to 70 | 15 to 65 | 20 to 60 |
| formula (1) | | | | |
| Non-ionic surfactant | 5 to 50 | 10 to 45 | 15 to 40 | 20 to 35 |
| Phosphonate | 0.5 to 10 | 1 to 8 | 1 to 8 | 3 to 6 |
| Misc. | up to 100 | up to 100 | up to 100 | up to 100 |

A process for cleaning surfaces, in the course of which a compound of general formula (1) or a cleaning agent containing a compound of general formula (1) is introduced into a cleaning liquor, is an additional subject matter of this application.

Compounds of general formula (1) can be prepared by neutralization of a corresponding sulfonic acid with the corresponding amine. A process for preparing a compound of general formula (1), comprising the steps of
1) providing a sulfonic acid of general formula R—X—SO₃H (2), wherein
i) R is selected from the group of C₆ to C₂₀ alkyl functional groups;
ii) X is selected from the group
a) C₆H₄
b) (OC₂H₄)ₙO where n=2 to 8
c) C(O)—N(H₃)CH₂H₂
d) R¹O(O)CCH₂(R¹O(O)C)CH, in which R¹═C₇ to C₂₀ alkyl
2) providing an amine of general formula N(RₐR_{b}R_{c}) (3), in which at least two of the functional groups Rₐ, R_{b}, and R_{c} are selected from the group consisting of C₃ to C₁₂ alkyl and C₃ to C₁₂ hydroxyalkyl functional groups;
3) mixing the sulfonic acid (2) and the amine (3) to form a compound of general formula (1)
is another subject matter of this application.

Compounds of general formula (1) can, as described above, be prepared by reacting a sulfonic acid (2) with a corresponding amine (3). Preferred detergents based upon compounds of general formula (1) can thus be described in other words by their content of sulfonic acid (2) and amine (3). The composition of some preferred detergents or cleaning agents can be derived from the following tables (amounts given in wt. % based on the total weight of the preparation, unless otherwise indicated). The listed detergent or cleaning agents are free of fluorine-containing hydrocarbon solvents.

| | Formula 21 | Formula 22 | Formula 23 | Formula 24 |
|---|---|---|---|---|
| R—X—SO₃H (2) ¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Misc. | up to 100 | up to 100 | up to 100 | up to 100 |
| | Formula 25 | Formula 26 | Formula 27 | Formula 28 |
| R—X—SO₃H (2) ¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Enzyme preparation | 0.2 to 12 | 0.5 to 10 | 0.5 to 10 | 1 to 8 |
| Misc. | up to 100 | up to 100 | up to 100 | up to 100 |
| | Formula 31 | Formula 32 | Formula 33 | Formula 34 |
| R—X—SO₃H (2) ¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Wash- or cleaning- | 0.1 to 14 | 0.2 to 12 | 0.5 to 10 | 0.5 to 10 |
| active polymer | | | | |
| Misc. | up to 100 | up to 100 | up to 100 | up to 100 |
| | Formula 35 | Formula 36 | Formula 37 | Formula 38 |
| R-X-SO₃H (2) ¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Phosphonate | 0.5 to 10 | 1 to 8 | 1 to 8 | 3 to 6 |
| Misc. | up to 100 | up to 100 | up to 100 | up to 100 |
| | Formula 41 | Formula 42 | Formula 43 | Formula 44 |
| R—X—SO₃H (2) ¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Fatty acid | 1 to 12 | 1 to 12 | 2 to 10 | 2 to 10 |
| Misc. | up to 100 | up to 100 | up to 100 | up to 100 |
| | Formula 45 | Formula 46 | Formula 47 | Formula 48 |
| R—X—SO₃H (2) ¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Enzyme preparation | 0.2 to 12 | 0.5 to 10 | 0.5 to 10 | 1 to 8 |
| Water | <10 | <10 | <7 | <5 |
| Misc. | up to 100 | up to 100 | up to 100 | up to 100 |
| | Formula 51 | Formula 52 | Formula 53 | Formula 54 |
| R—X—SO₃H (2) ¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Enzyme preparation | 0.2 to 12 | 0.5 to 10 | 0.5 to 10 | 1 to 8 |
| Fatty acid | 1 to 12 | 1 to 12 | 2 to 10 | 2 to 10 |
| Water | <10 | <10 | <7 | <5 |
| Misc. | up to 100 | up to 100 | up to 100 | up to 100 |
| | Formula 55 | Formula 56 | Formula 57 | Formula 58 |
| R—X—SO₃H (2) ¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Enzyme preparation | 0.2 to 12 | 0.5 to 10 | 0.5 to 10 | 1 to 8 |
| Fatty acid | 1 to 12 | 1 to 12 | 2 to 10 | 2 to 10 |
| Organic solvent | 3 to 24 | 6 to 22 | 6 to 22 | 10 to 20 |
| Water | <10 | <10 | <7 | <5 |
| Misc. | up to 100 | up to 100 | up to 100 | up to 100 |
| | Formula 61 | Formula 62 | Formula 63 | Formula 64 |
| R—X—SO₃H (2) ¹ | 5 to 25 | 5 to 25 | 10 to 20 | 10 to 20 |
| Amine (3) | 5 to 25 | 5 to 25 | 10 to 20 | 10 to 20 |
| Non-ionic surfactant | 5 to 50 | 10 to 45 | 15 to 40 | 20 to 35 |
| Enzyme preparation | 0.2 to 12 | 0.5 to 10 | 0.5 to 10 | 1 to 8 |
| Fatty acid | 1 to 12 | 1 to 12 | 2 to 10 | 2 to 10 |
| Wash- or cleaning- | 0.1 to 14 | 0.2 to 12 | 0.5 to 10 | 0.5 to 10 |
| active polymer | | | | |
| Organic solvent | 3 to 24 | 6 to 22 | 6 to 22 | 10 to 20 |
| Water | <10 | <10 | <7 | <5 |
| Misc. | up to 100 | up to 100 | up to 100 | up to 100 |

¹ X is selected from the group

a) C₆H₄
b) (OC₂H₄)ₙO where n=2 to 8
c) C(O)—N(H₃)CH₂CH₂
d) R¹O(O)CCH₂(R¹O(O)C)CH, in which R¹═C₇ to C₂₀ alkyl
preferably from C₆H₄

The compounds of general formula (1) are particularly suitable for the preparation of detergent compositions with a high detergent content. In addition to their high content of sulfonic acid (2) neutralized with amine (3), these compositions are also characterized in particular by a low solvent content. The composition of some preferred highly-concentrated detergent or cleaning agent product forms can be derived from the following tables (amounts given in wt. % based on the total weight of the preparation, unless otherwise indicated). The listed detergent or cleaning agents are free of fluorine-containing hydrocarbon solvents.

| | Formula 71 | Formula 72 | Formula 73 | Formula 74 |
|---|---|---|---|---|
| R—X—SO₃H (2) ² | 20 to 40 | 20 to 40 | 25 to 35 | 25 to 35 |
| Amine (3) | 25 to 50 | 30 to 50 | 35 to 50 | 35 to 45 |
| Non-ionic surfactant | 0 to 15 | 0 to 15 | 2 to 12 | 4 to 10 |
| Misc. | up to 100 | up to 100 | up to 100 | up to 100 |
| | Formula 75 | Formula 76 | Formula 77 | Formula 78 |
| R—X—SO₃H (2) ² | 20 to 40 | 20 to 40 | 25 to 35 | 25 to 35 |
| Amine (3) | 25 to 50 | 30 to 50 | 35 to 50 | 35 to 45 |
| Non-ionic surfactant | 0 to 15 | 0 to 15 | 2 to 12 | 4 to 10 |
| Enzyme preparation | 0.2 to 12 | 0.5 to 10 | 0.5 to 10 | 1 to 8 |
| Misc. | up to 100 | up to 100 | up to 100 | up to 100 |
| | Formula 81 | Formula 82 | Formula 83 | Formula 84 |
| R—X—SO₃H (2) ² | 20 to 40 | 20 to 40 | 25 to 35 | 25 to 35 |
| Amine (3) | 25 to 50 | 30 to 50 | 35 to 50 | 35 to 45 |
| Non-ionic surfactant | 0 to 15 | 0 to 15 | 2 to 12 | 4 to 10 |
| Wash- or cleaning- | 0.1 to 14 | 0.2 to 12 | 0.5 to 10 | 0.5 to 10 |
| active polymer | | | | |
| Misc. | up to 100 | up to 100 | up to 100 | up to 100 |
| | Formula 85 | Formula 86 | Formula 87 | Formula 88 |
| R—X—SO₃H (2) ² | 20 to 40 | 20 to 40 | 25 to 35 | 25 to 35 |
| Amine (3) | 25 to 50 | 30 to 50 | 35 to 50 | 35 to 45 |
| Non-ionic surfactant | 0 to 15 | 0 to 15 | 2 to 12 | 4 to 10 |
| Phosphonate | 0.5 to 10 | 1 to 8 | 1 to 8 | 3 to 6 |
| Misc. | up to 100 | up to 100 | up to 100 | up to 100 |
| | Formula 91 | Formula 92 | Formula 93 | Formula 94 |
| R—X—SO₃H (2) ² | 20 to 40 | 20 to 40 | 25 to 35 | 25 to 35 |
| Amine (3) | 25 to 50 | 30 to 50 | 35 to 50 | 35 to 45 |
| Non-ionic surfactant | 0 to 15 | 0 to 15 | 2 to 12 | 4 to 10 |
| Fatty acid | 1 to 12 | 1 to 12 | 2 to 10 | 2 to 10 |
| Misc. | up to 100 | up to 100 | up to 100 | up to 100 |
| | Formula 95 | Formula 96 | Formula 97 | Formula 98 |
| R—X—SO₃H (2) ² | 20 to 40 | 20 to 40 | 25 to 35 | 25 to 35 |
| Amine (3) | 25 to 50 | 30 to 50 | 35 to 50 | 35 to 45 |
| Non-ionic surfactant | 0 to 15 | 0 to 15 | 2 to 12 | 4 to 10 |
| Enzyme preparation | 0.2 to 12 | 0.5 to 10 | 0.5 to 10 | 1 to 8 |
| Water | <5 | <5 | <3 | <3 |
| Misc. | up to 100 | up to 100 | up to 100 | up to 100 |
| | Formula 101 | Formula 102 | Formula 103 | Formula 104 |
| R—X—SO₃H (2) ² | 20 to 40 | 20 to 40 | 25 to 35 | 25 to 35 |
| Amine (3) | 25 to 50 | 30 to 50 | 35 to 50 | 35 to 45 |
| Non-ionic surfactant | 0 to 15 | 0 to 15 | 2 to 12 | 4 to 10 |
| Enzyme preparation | 0.2 to 12 | 0.5 to 10 | 0.5 to 10 | 1 to 8 |
| Fatty acid | 1 to 12 | 1 to 12 | 2 to 10 | 2 to 10 |
| Water | <5 | <5 | <3 | <3 |
| Misc. | up to 100 | up to 100 | up to 100 | up to 100 |
| | Formula 105 | Formula 106 | Formula 107 | Formula 108 |
| R—X—SO₃H (2) ² | 20 to 40 | 20 to 40 | 25 to 35 | 25 to 35 |
| Amine (3) | 25 to 50 | 30 to 50 | 35 to 50 | 35 to 45 |
| Non-ionic surfactant | 0 to 15 | 0 to 15 | 2 to 12 | 4 to 10 |
| Enzyme preparation | 0.2 to 12 | 0.5 to 10 | 0.5 to 10 | 1 to 8 |
| Fatty acid | 1 to 12 | 1 to 12 | 2 to 10 | 2 to 10 |
| Organic solvent | <5 | <5 | <3 | <3 |
| Water | <5 | <5 | <3 | <3 |
| Misc. | up to 100 | up to 100 | up to 100 | up to 100 |
| | Formula 111 | Formula 112 | Formula 113 | Formula 114 |
| R—X—SO₃H (2) ² | 20 to 40 | 20 to 40 | 25 to 35 | 25 to 35 |
| Amine (3) | 25 to 50 | 30 to 50 | 35 to 50 | 35 to 45 |
| Non-ionic surfactant | 0 to 15 | 0 to 15 | 2 to 12 | 4 to 10 |
| Enzyme preparation | 0.2 to 12 | 0.5 to 10 | 0.5 to 10 | 1 to 8 |
| Fatty acid | 1 to 12 | 1 to 12 | 2 to 10 | 2 to 10 |
| Wash- or cleaning- | 0.1 to 14 | 0.2 to 12 | 0.5 to 10 | 0.5 to 10 |
| active polymer | | | | |
| Organic solvent | <5 | <5 | <3 | <3 |
| Water | <5 | <5 | <3 | <3 |
| Misc. | up to 100 | up to 100 | up to 100 | up to 100 |

² X is selected from the group

a) C₆H₄
b) (OC₂H₄)ₙO where n=2 to 8
c) C(O)—N(CH₃)CH₂CH₂
d) R¹O(O)CCH₂(R¹O(O)C)CH, in which R¹═C₇ to C₂₀ alkyl
preferably from C₆H₄

The detergent or cleaning agents described above are preferably free of quaternary amines and organic pigments.

This application provides, inter alia, the following subject matter:

1. A compound of general formula (1)

R—X—SO₃⁻HN⁺(RₐR_{b}R_{c}) (1), where
i) R is selected from the group of C₆ to C₂₀ alkyl functional groups;
ii) X is selected from the group
a) C₆H₄
b) b) (OC₂H₄)ₙO where n=2 to 8
c) c) C(O)—N(CH₃)CH₂CH₂
d) d) R¹O(O)CCH₂(R¹O(O)C)CH, in which R¹═C₆ to C₂₀ alkyl
iii) at least two of the functional groups Rₐ, R_{b}, and R_{c} are selected independently of each other from the group consisting of C₃ to C₁₂ alkyl and C₃ to C₁₂ hydroxyalkyl functional groups.

2. The compound of general formula (1) according to point 1, wherein
i) R is selected from the group of C₁₀ to C₁₃ alkyl functional groups;
ii) X is selected from group C₆H₄
iii) at least two of the functional groups Rₐ, R_{b}, and R_{c} are selected independently of each other from the group consisting of C₃ to C₁₂ alkyl and C₃ to C₁₂ hydroxyalkyl functional groups.

3. The compound of general formula (1) according to point 2, wherein
i) R is selected from the group of linear C₁₀ to C₁₃ alkyl functional groups.

4. The compound of general formula (1) according to one of points 2 or 3, wherein
i) R is selected from the group of secondary C₁₀ to C₁₃ alkyl functional groups.

5. The compound of general formula (1) according to one of points 2 to 4, wherein
i) R is selected from the group of linear secondary C₁₀ to C₁₃ alkyl functional groups.

6. The compound of general formula (1) according to one of points 2 to 5, wherein
i) R represents a linear C₁₂—H₂₅ alkyl functional group.

7. The compound of general formula (1) according to one of points 2 to 6, wherein
i) R represents a linear secondary C₁₂H₂₅ alkyl functional group.

8. The compound of general formula (1) according to point 1, wherein
i) R is selected from the group of C₁₂ to C₂₀ alkyl functional groups;
ii) X is selected from the group selected from the group (OC₂H₄)ₙO where n=1 to 8
iii) at least two of the functional groups Rₐ, R_{b}, and R_{c} are selected independently of each other from the group consisting of C₃ to C₁₂ alkyl and C₃ to C₁₂ hydroxyalkyl functional groups.

9. The compound of general formula (1) according to point 1, wherein
i) R is selected from the group of C₇ to C₁₇ alkyl functional groups;
ii) X is selected from the group C(O)—N(CH₃)CH₂CH₂;
iii) at least two of the functional groups Rₐ, R_{b}, and R_{c} are selected independently of each other from the group consisting of C₃ to C₁₂ alkyl and C₃ to C₁₂ hydroxyalkyl functional groups.

10. The compound of general formula (1) according to point 1, wherein
i) R is selected from the group of C₇ to C₁₇ alkyl functional groups;
ii) X is selected from the group O(O)CCH₂(RO(O)C)CH where R═C₆ to C₁₇ alkyl;
iii) at least two of the functional groups Rₐ, R_{b}, and R_{c} are selected independently of each other from the group consisting of C₃ to C₁₂ alkyl and C₃ to C₁₂ hydroxyalkyl functional groups.

11. The compound of general formula (1) according to one of the preceding points, wherein
i) at least two of the functional groups Rₐ, R_{b}, and R_{c} are selected independently of each other from the group consisting of C₃ to C₁₂ alkyl functional groups, preferably from the group consisting of C₃ to C₆ alkyl functional groups.

12. The compound of general formula (1) according to one of the preceding points, wherein
iii) the three functional groups Rₐ, R_{b}, and R_{c} are selected independently of each other from the group consisting of C₃ to C₁₂ alkyl functional groups, preferably from the group consisting of C₃ to C₆ alkyl functional groups.

13. The compound of general formula (1) according to one of points 1 to 10, wherein
iii) at least two of the functional groups Rₐ, R_{b}, and R_{c} are selected independently of each other from the group consisting of C₃ to C₁₂ hydroxyalkyl functional groups, preferably from the group consisting of C₃ to C₆ hydroxyalkyl functional groups.

14. The compound of general formula (1) according to one of points 1 to 10 or 13, wherein
iii) the three functional groups Rₐ, R_{b}, and R_{c} are selected independently of each other from the group consisting of C₃ to C₁₂ hydroxyalkyl functional groups, preferably from the group consisting of C₃ to C₆ hydroxyalkyl functional groups.

15. The compound of general formula (1) according to one of points 1 to 10, 13, or 14, wherein
iii) the three functional groups Rₐ, R_{b}, and R_{c} each represent a (CH₂CH(OH)CH₃) hydroxyalkyl functional group.

16. The compound of general formula (1) according to point 1, wherein
i) R represents a linear C₁₂H₂₅ alkyl functional group;
ii) X is selected from group C₆H₄
iii) Rₐ, R_{b}, and R_{c} each represent a (CH₂CH(OH)CH₃) hydroxyalkyl functional group.

17. The compound of general formula (1) according to point 1, wherein
i) R represents a linear secondary C₁₂H₂₅ alkyl functional group;
ii) X is selected from group C₆H₄
iii) Rₐ, R_{b}, and R_{c} each represent a (CH₂CH(OH)CH₃) hydroxyalkyl functional group.

18. A detergent or cleaning agent containing a compound of general formula (1) according to one of the preceding points.

19. A liquid detergent or cleaning agent containing a compound of general formula (1) according to one of the preceding points.

20. The detergent or cleaning agent according to one of points 18 or 19, wherein the detergent or cleaning agent is free of fluorine-containing hydrocarbon solvents.

21. A process for washing textiles, over the course of which a compound of general formula (1) according to one of points 1 to 17 or a detergent or cleaning agent according to point 18 is introduced into a washing liquor or is applied to a textile surface.

22. The process according to point 21, wherein the washing liquor is free of fluorine-containing hydrocarbon solvents.

23. The process according to one of points 21 or 22, wherein the washing liquor is aqueous.

24. A process for cleaning hard surfaces, over the course of which a compound of general formula (1) according to one of the preceding points 1 to 17 or a detergent or cleaning agent according to point 18 is introduced into a cleaning liquor or is applied to a hard surface.

25. The process according to point 24, wherein the cleaning liquor is free of fluorine-containing hydrocarbon solvents.

26. The process according to one of points 24 or 25, wherein the cleaning liquor is aqueous.

27. A process for preparing a compound of general formula (1) according to one of points 1 to 17, comprising the steps of
1) providing a sulfonic acid of general formula R—X—SO₃H (2), wherein
i) R is selected from the group of C₆ to C₂₀ alkyl functional groups;
ii) X is selected from the group
a) C₆H₄
b) (OC₂H₄)ₙO where n=2 to 8
c) C(O)—N(CH₃)CH₂CH₂
d) R¹O(O)CCH₂(R¹O(O)C)CH, in which R═C₇ to C₂₀ alkyl
2) providing an amine of general formula N(RₐR_{b}R_{c}) (3), in which at least two of the functional groups Rₐ, R_{b}, and R_{c} are selected from the group consisting of C₃ to C₁₂ alkyl and C₃ to C₁₂ hydroxyalkyl functional groups;
3) mixing the sulfonic acid (2) and the amine (3) to form a compound of general formula (1).

### Examples

Equimolar amounts of dodecylbenzene sulfonic acid and triisopropanolamine are reacted with each other in a reaction vessel. A transparent, viscous, surface-active ionic liquid of the formula C₁₂H₂₅—C₆H₄—SO₃⁻HN⁺(CH₂CH(OH)CH₃)₃ is formed.

The neutralization products of dodecylbenzene sulfonic acid with triisopropanolamine (according to the invention) and monoisopropanolamine (not according to the invention) were investigated with regard to their optical properties using

| C₁₂H₂₅—C₆H₄—SO₃⁻HN⁺(CH₂CH(OH)CH₃)₃ | C₁₂H₂₅—C₆H₄—SO₃⁻H₃N⁺CH₂CH(OH)CH₃ |
|---|---|
| amorphous, isotropic | liquid crystalline, anisotropic |

The rheological investigation of the neutralization products of dodecylbenzene sulfonic acid with triisopropanolamine (according to the invention) and monoisopropanolamine (not according to the invention) gave the following results:

| C₁₂H₂₅—C₆H₄—SO₃⁻HN⁺(CH₂CH(OH)CH₃)₃ | C₁₂H₂₅—C₆H₄—SO₃⁻H₃N⁺CH₂CH(OH)CH₃ |
|---|---|
| no pour point | pour point |
| viscosity independent of shear rate | viscosity dependent upon shear rate |

Thermal analysis by differential scanning calorimetry (DSC) of the neutralization products of dodecylbenzene sulfonic acid with triisopropanolamine (according to the invention) and monoisopropanolamine (not according to the invention) gave the following result:

| C₁₂H₂₅—C₆H₄—SO₃⁻HN⁺(CH₂CH(OH)CH₃)₃ | C₁₂H₂₅—C₆H₄—SO₃⁻H₃N⁺CH₂CH(OH)CH₃ |
|---|---|
| glass transition temperature | glass transition temperature and melting |
| | point |

An exemplary detergent composition containing a compound of the general formula

| Ingredient | Wt. % |
|---|---|
| Ethoxylated C₁₂-C₁₈ fatty alcohol | 30 |
| (7EO) | |
| C₁₂H₂₅—C₆H₄—SO₃⁻HN⁺(CH₂CH(OH)CH₃)₃ | 48 |
| Polyalkoxylated amine ¹⁾ | 6 |
| Polyalkoxylated polyalkyleneimine ²⁾ | 6 |
| DTPMP | 5 |
| Misc. | up to 100 |

¹⁾ polyalkoxylated amine having a weight-average molecular weight Mw in the range of 600 g/mol to 10,000 g/mol, which is obtained by reacting ammonia or primary alkyl or hydroxyalkylamines having a molecular weight of less than 200 g/mol with alkylene oxides
²⁾ polyalkoxylated polyalkyleneimine which is obtained by reacting polyalkyleneimines with alkylene oxides.

What is claimed is:

1. A compound of general formula (1)
R—X—SO₃⁻HN⁺(RₐR_{b}R_{c}) (1), wherein
i.) R is selected from the group of C₆ to C₂₀ alkyl functional groups;
ii) X is selected from the group
a) C₆H₄
b) (OC₂H₄)ₙO where n=2 to 8
c) C(O)—N(CH₃)CH₂CH₂
d) R¹O(O)CCH₂(R¹O(O)C)CH, in which R¹═C₆ to C₂₀ alkyl
iii) at least two of the functional groups Rₐ, R_{b}, and R_{c} are selected independently of each other from the group consisting of C₃ to C₁₂ alkyl and C₃ to C₁₂ hydroxyalkyl functional groups.

2. The compound of general formula (1) according to claim 1, wherein
i) R is selected from the group of C₁₀ to C₁₃ alkyl functional groups;
ii) X is selected from group C₆H₄
iii) at least two of the functional groups Rₐ, R_{b}, and R_{c} are selected independently of each other from the group consisting of C₃ to C₁₂ alkyl and C₃ to C₁₂ hydroxyalkyl functional groups.

3. The compound of general formula (1) according to claim 1, wherein
i) R represents a linear secondary C₁₂H₂₅ alkyl functional group.

4. The compound of general formula (1) according to claim 1, wherein
i) the three functional groups Rₐ, R_{b}, and R_{c} are selected independently of each other from the group consisting of C₃ to C₁₂ hydroxyalkyl functional groups.

5. The compound of general formula (1) according to claim 1, wherein
i) R represents a linear C₁₂H₂₅ alkyl functional group;
ii) X is selected from group C₆H₄
iii) Rₐ, R_{b}, and R_{c} each represent a (CH₂CH(OH)CH₃) hydroxyalkyl functional group.

6. The compound of general formula (1) according to claim 1, wherein
i) R represents a linear secondary C₁₂H₂₅ alkyl functional group;
ii) X is selected from the group C₆H₄
iii) Rₐ, R_{b}, and R_{c} each represent a (CH₂CH(OH)CH₃) hydroxyalkyl functional group.

7. A detergent or cleaning agent containing a compound of general formula (1) according to claim 1.

8. A process for washing textiles, over the course of which a compound of general formula (1) according to claim 1 is introduced into a washing liquor or is applied to a textile surface.

9. A process for cleaning hard surfaces, over the course of which a compound of general formula (1) according to claim 1 is introduced into a cleaning liquor or is applied to a hard surface.

10. A process for preparing a compound of the general formula (1) according to claim 1, comprising the steps of
1) providing a sulfonic acid of general formula R—X—SO₃H (2), wherein
i) R is selected from the group of C₆ to C₂₀ alkyl functional groups;
ii) X is selected from the group
a) C₆H₄
b) (OC₂H₄)ₙO where n=2 to 8
c) C(O)—N(CH₃)CH₂CH₂
d) R¹O(O)CCH₂(R¹O(O)C)CH, in which R═C₆ to C₂₀ alkyl
2) providing an amine of general formula N(RₐR_{b}R_{c}) (3), in which at least two of the functional groups Rₐ, R_{b}, and R_{c} are selected from the group consisting of C₃ to C₁₂ alkyl and C₃ to C₁₂ hydroxyalkyl functional groups;
3) mixing the sulfonic acid (2) and the amine (3) to form a compound of general formula (1).
